(19) United States
(12) Patent Application Publication
(10) Pub. No.: US 2006/0206070 A1
(43) Pub. Date: Sep. 14, 2006

(54) Hygienic means

(76) Inventor: Andreo Larsen, Helsinki (FI)

Correspondence Address: COOPER & DUNHAM, LLP, 1185 AVENUE OF THE AMERICAS, NEW YORK, NY, 10036, US

(21) Appl. No.: 10/566,347

(22) PCT Filed: Jul. 27, 2004
(86) PCT No.: PCT/FI04/00463

§ 371 Date: Jan. 26, 2006

(30) Foreign Application Priority Data
Jul. 28, 2003 (FI) 20031105

(51) Int. Cl. A61F 5/44

(52) U.S. Cl. 604/354; 604/385.09; 604/347

(57) ABSTRACT

The invention relates to a male hygienic means intended as dribble protection after urination. The means comprises a sheath (1) preferably containing an absorbent material to be fitted at the tip of the penis, fixing strips (2) with adhesive surfaces on opposite sides of the sheath, and a rip tag (7) protecting the adhesive surfaces before the means is fixed. In accordance with the invention, the principally U-shaped rip tag (7) extends around the front side of the sheath tip and its opposite ends (8) adhere to the laterally bent fixing strips (2) of the sheath (1). The user may fix the means into position by pushing his fingers between the opposite sides of the sheath (1) and the rip tag (7), so that with one single pulling motion, he can fix the fixing strips to the flanks of his penis and release the rip tag.

This invention relates to a hygienic means, comprising a sheath to be fitted on the tip of the penis and fixing strips with adhesive surfaces, which have been bent to opposite sides of the sheath, the adhesive surfaces of both of the strips having been protected with a rip tag, allowing the fixing strips to be pulled along the flanks of the penis as the means is being fixed into position.

After each urination, some additional urine dribbles are typically secreted from the male penis. Prostate disorders, such as infections and prostatic hypertrophy, which are most common especially in elderly men, increase the risk of dribbling and of moderate urinary incontinence. However, such minor dribbles occur after urination also in quite healthy men.

There are items intended for female urinary incontinence and similar personal hygiene disorders. Sanitary pads for women have become widely popular worldwide even among symptomless women.

There are protective items for male urinary incontinence that resolve major incontinence problems relating e.g. to post-surgical conditions, diseases or lesions. Such means are typically bulky, have large retention capacity and are awkward to fit into position, and their destruction is also difficult.

So far, there have been no hygienic items for men similar to those sold on a mass scale for women. Consequently, there is an obvious demand in the market for a simple personal hygiene item produced in mass production, which would avoid hygienic problems and fouled garments caused by dribbling after urination. For the item to be accepted among consumers, it should meet the following requirements: small size, ease of use, reliably retention in position, convenient destruction and adequate price.

The patent literature discloses various urinary incontinence protective items for men, most of which are intended for patients in institutional care. U.S. Pat. No. 4,863,448 (Berg) discloses a more straightforward hygienic means for use as a dribble protector, one embodiment of which comprises fixing strips with adhesive surfaces on the sides of the sheath portion, the strips being protected with an annular tear-off portion surrounding the sheath. When this portion is compressed at its ends, the sheath opens and can be fitted in position around the tip of the penis. The tear-off portion is then pulled back so as to be released from the fixing strips while the strip adheres to the flanks of the penis under the pulling movement, cf. FIGS. 13-14 of the reference. This solution involves the problem that, for removal from around the penis, the annular tear-off portion eventually requires to be torn transversely.

U.S. Pat. No. 4,790,835 (Elias) depicts a hygienic means, in which part of the sheath has been formed as a cut fixing strip with an adhesive surface. The sheath is pressed into position by pulling the fixing strip laterally and by adhesion to the flank of the sheath, the sheath and the strip being pulled so as to overlap in the attachment area. In the applicant's view, this solution does not either meet the requirements of adequate comfort and ease of use posed on the item.

U.S. Pat. No. 4,601,716 (smith) describes a bag-like male dribble protector, which has to be tightened into position by means of a separate fixing strip with an adhesive surface protruding from the side of the bag. This item is bulky and its comfort of use is doubtful in the applicant's opinion.

The object of the invention is to provide a hygienic means intended as a male dribble protective means, which resolves the problems above owing to the sufficiently straightforward design and ease of use of the item. The invention is characterised by the sheath-like means comprising a uniform, substantially U-shaped rip tag, which extends around the front side of the sheath tip and is fixed at its opposite ends to the adhesive surfaces of the fixing strips.

In the hygienic means of the invention, the fixing strips on its opposite sides have been protected with one single common rip tag, which, when peeled off, releases the adhesive surfaces of the strips so that they can be fixed by pressing against the flanks of the penis. The rip tag is released in the form of an elongated band, without causing any problem of removal similar to that of U.S. Pat. No. 4,863,448.

Since the sheath acts primarily as a urinary incontinence protector, it preferably comprises an absorbent material.

In a preferred embodiment of the invention, the end of the U-shaped rip tag has been bent inwardly against the adhesive surface of the fixing strip. This allows the rip tag to be readily released from the adhesive surface by peeling as it is pulled in the longitudinal direction from the tip of the sheath outwardly, or as it is pushed from the inside of the tag outwardly from the sheath flank. Preferably the U-shaped rip tag has been bent inwardly, identically at both ends, against the adhesive surfaces of the fixing strips provided on opposite sides if the sheath.

In the embodiment mentioned above, the U-shaped rip tag can be particularly advantageously dimensioned such that, on both sides of the sheath, between the sheath and the rip tag, a gap is formed large enough to receive a finger, allowing the user to release the adhesive surfaces of the fixing strips from the rip tag by the same pulling motion as the one performed for straightening the fixing strips in fixing them into position. In other words, with one single finger pulling motion for pressing the fixing strips against the penis flanks, the user simultaneously peels off the ends of the rip tag from the adhesive surfaces of the fixing strips, without needing any other action for removing the rip tag.

In addition, the rip tag can be equipped with a fold, pleat or like gripping point at its centre, at the sheath tip, and by pulling at this the user peels off the tag ends from the adhesive surfaces of the fixing strips, or by gripping this he may facilitate the attachment of the strips with his fingers as mentioned above.

It is further possible to extend the end of the rip tag over the adhesive surface of the fixing strip such that it forms a free gripping point, the user peeling off the rip tag from the adhesive surface by pulling at this point. The tag is more readily peeled off if its opposite end is bent inwardly and fixed to the fixing strip provided on the opposite side of the sheath, as explained above.

In a preferred embodiment of the invention, the fixing strip comprises a tip portion not covered with adhesive coating, which forms a gripping point, and by pulling at this the user can remove the means after use.

The invention is explained in greater detail below by means of examples and with reference to the accompanying drawings, in which

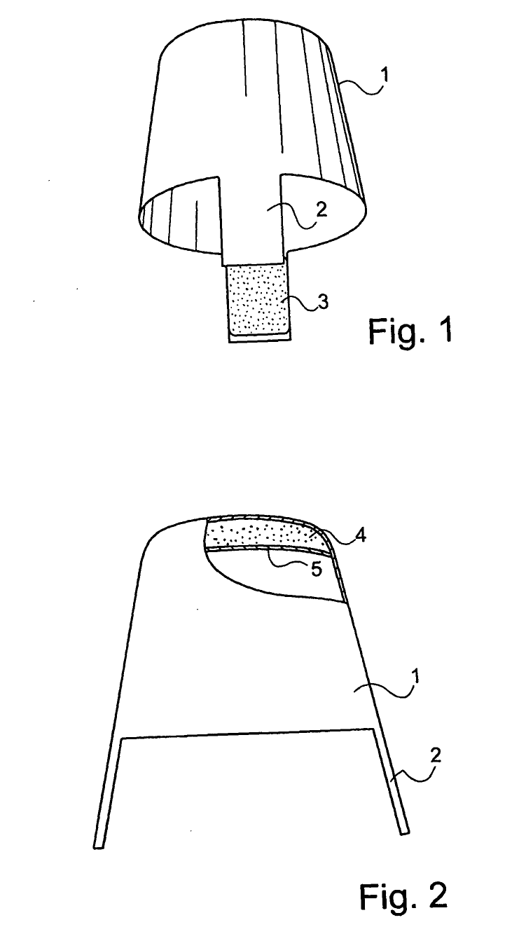

FIG. 1 shows a hygienic means of the invention, which is intended as a male dribble protector and has no rip tag protecting its adhesive surfaces,

FIG. 2 is a lateral projection of the means of FIG. 1 with its tip partially cut,

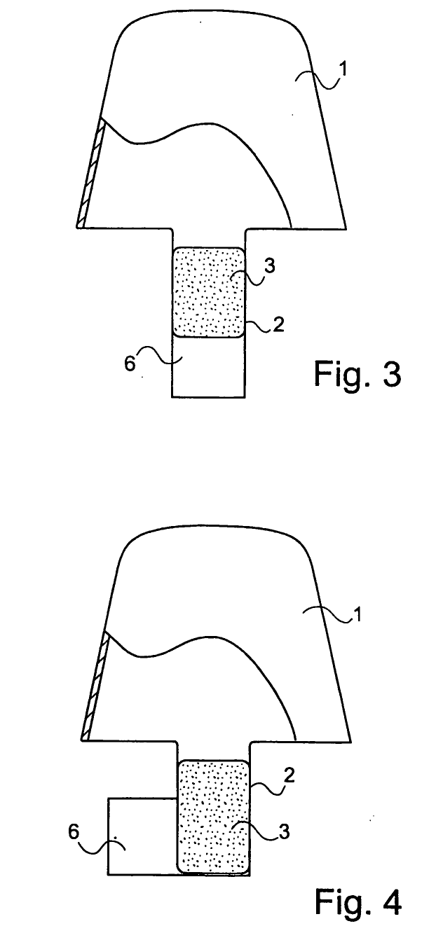

FIG. 3 a hygienic means of another embodiment of the invention in partially cut state,

FIG. 4 corresponding to FIG. 3 shows a hygienic means of a third embodiment of the invention,

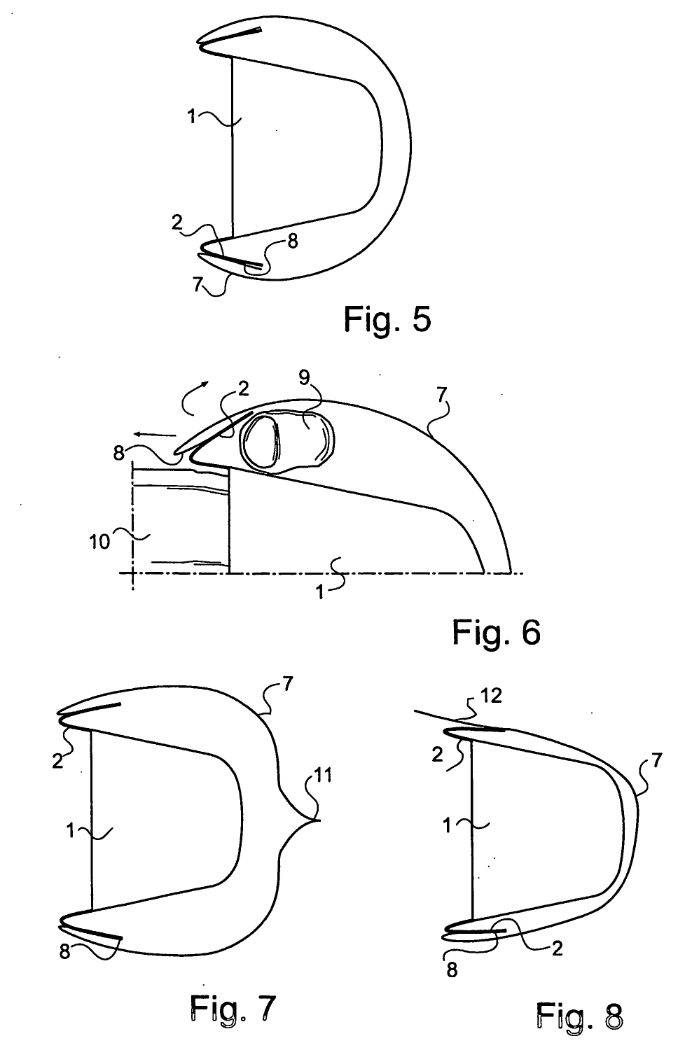

FIG. 5 is a lateral projection of the hygienic means of FIG. 1 including the rip tag, before the means is fixed into position,

FIG. 6 illustrates the fixing of the means of FIG. 5 with the user pushing the fixing strips into position with his fingers,

FIG. 7 corresponding to FIG. 5 shows the hygienic means of FIG. 1 equipped with a rip tag having a pleat for peeling off the tag, and

FIG. 8 corresponding to FIGS. 5 and 7 shows the hygienic means equipped with a rip tag extended over the fixing strip.

The hygienic means illustrated in FIGS. 1 and 2 is a dribble protector fixed to the male penis after urination with the task to protect cloths from dribbles after urination or against moderate urinary incontinence. The means generally comprises a conically shaped sheath 1 and fixing strips 2 extending the sheath mantle on both sides of the means, the inner surfaces of the fixing strips being equipped with an adhesive coating 3.

The sheath 1 and the fixing strips 2 are made from one piece, preferably of a biodegradable material, such as e.g. polymer-coated hydrophobic paper. The sheath 1 in FIG. 2 has a tip portion made of an absorbent, e.g. cellulose-based fibre material 4, while the inner surface 5 of the sheath is made of a porous liquid-permeable paper.

In the embodiment of FIG. 3, the fixing strip 2 comprises a tip portion 6 not covered with adhesive coating 3, the user being able to release the means from its position around the penis by pulling at this after use. It is also possible for the user to detach only one side of the means remaining in position, and to turn it aside while urinating, and then to fit the means anew and fix it with the aid of the fixing strip. Consequently, the user does not have to replace the means each time he urinates.

The means illustrated in FIG. 4 corresponds to that of FIG. 3, except that the tip 6 of the fixing strip 2, at which the user pulls for releasing the strip, is located on the side of the adhesive surface 3, the strip forming a 90° angle.

FIG. 5 shows the hygienic means of FIG. 1 equipped with a rip tag 7, which protects the adhesive surfaces 3 of the fixing strips 2 before the means is fixed into position. At this stage, the fixing strips 2 are bent to the opposite sides of the sheath 1, and the ends 8 of the principally U-shaped uniform rip tag 7 extending around the front side of the sheath 1 are bent inwardly against the adhesive surfaces of the fixing strips 2 facing away from the mantle of the sheath 1.

While using the means, the user fits the tip of his penis into the sheath 1 with the rip tag 7 protecting the adhesive surfaces of the fixing strips 2, as shown in FIG. 5. The means is subsequently attached in the manner shown in FIG. 6. The user pushes his finger 9 into the space between the sheath 1 and the sufficiently loosely dimensioned rip tag 7 on both sides of the sheath 1 (FIG. 6 shows only one side of the means), and then the user, with one single backward pulling movement, straightens the fixing strips 2 so that their adhesive surfaces 3 are pressed against the flanks of the penis 10, at the same time as the bent ends 8 of the rip tag 7 are released by peeling from the adhesive surfaces 3 as the fingers 9 proceed. The rip tag 7 is thus removed at the same time as the means is attached without any separate actions.

The embodiment of the invention shown in FIG. 7 differs from that of FIG. 5 principally by the feature that a flat pleat 11 has been formed in front of the sheath 1, generally at the centre of the U-shaped rip tag 7, the user being able to detach the rip tag by pulling at the pleat or to grip the pleat of the rip tag while attaching the means, as illustrated in FIG. 6.

In the embodiment of the invention shown in FIG. 8, the inwardly bent end 8 of the U-shaped rip tag 7 is fixed to the adhesive surface 3 of the fixing strip 2, in the manner explained in conjunction with FIG. 5, whereas the opposite end 12 of the rip tag has been extended on the opposite side of the means to pass by the fixing strip 2, allowing the user to pull this end backwardly, i.e. in the direction of the tip of the sheath 1, in order to release the rip tag from the fixing strips 2. After this, the user can fix the fixing strips 2 with their released adhesive surfaces separately into position by pressing.

It is obvious to those skilled in the art that the applications of the invention are not confined to the examples described in detail above, but may vary within the scope of the following examples.

1. A hygienic means comprising a sheath (1) containing absorbent material (4) to be fitted on the tip of the penis (10) and fixing strips (2) with adhesive surfaces bent to opposite sides of the sheath, the adhesive surfaces (3) of both the fixing strips being protected with a strippable rip tag (7), allowing the user to pull the fixing strips against the flanks of his penis while fixing the means into position, characterised in that the means comprises a uniform, substantially U-shaped rip tag (7) extending around the front side of the tip of the sheath (1) and fixed at its opposite ends (8) to the adhesive surfaces (3) of the fixing strips (2).

2. A hygienic means as defined in claim 1, characterised in that the sheath (1) contains an absorbent material (4).

3. A hygienic means as defined in claim 1, characterised in that the end (8) of the U-shaped rip tag (7) is bent inwardly against the adhesive surface (3) of the fixing strip (2).

4. A hygienic means as defined in claim 3, characterised in that both the ends (8) of the U-shaped rip tag (7) are bent inwardly against the adhesive surfaces (3) of the fixing strips (2).

5. A hygienic means as defined in claim 4, characterised in that the rip tag (7) has been dimensioned such that a gap large enough for receiving the user's finger (9) forms on both sides if the sheath (1), between the sheath and the rip tag, the adhesive surfaces (3) of the fixing strips (2) being released from the rip tag by the same pulling motion by means of which the user straightens the fixing strips while fixing them into position.

6. A hygienic means as defined in claim 4, characterised in that a protruding fold or like gripping point (11) has been formed at the centre of the rip tag (7), at the tip of the sheath (1), the user being able to release the rip tag from the fixing strips (2) by pulling at the fold.

7. A hygienic means as defined in claim 1, characterised in that the end (12) of the rip tag (7) extends over the adhesive surface (3) of the fixing strip (2) , forming a gripping point allowing the user to release the rip tag from the fixing strip by pulling at this gripping point.

8. A hygienic means as defined in claim 1, characterised in that the fixing strip (2) has a tip portion (6) not covered with adhesive coating (3), allowing the user to remove the means after use by pulling at this tip portion.

9. A hygienic means as defined in claim 1, characterised in that the sheath (1) and the fixing strips (2) are formed of a uniform piece made of the same material.

10. A hygienic means as defined in claim 1, characterised in that the sheath (1) is made of a fibre-based material such as paper.

11. A hygienic means as defined in claim 2, characterised in that an absorbent fibre material (4) has been fitted within the sheath (1).

12. A hygienic means as defined in claim 2, characterised in that the end (8) of the U-shaped rip tag (7) is bent inwardly against the adhesive surface (3) of the fixing strip (2).

13. A hygienic means as defined in claim 12, characterised in that both the ends (8) of the U-shaped rip tag (7) are bent inwardly against the adhesive surfaces (3) of the fixing strips (2).

14. A hygienic means as defined in claim 13, characterised in that the rip tag (7) has been dimensioned such that a gap large enough for receiving the user's finger (9) forms on both sides if the sheath (1), between the sheath and the rip tag, the adhesive surfaces (3) of the fixing strips (2) being released from the rip tag by the same pulling motion by means of which the user straightens the fixing strips while fixing them into position.

15. A hygienic means as defined in claim 13, characterised in that a protruding fold or like gripping point (11) has been formed at the centre of the rip tag (7), at the tip of the. sheath (1), the user being able to release the rip tag from the fixing strips (2) by pulling at the fold.

16. A hygienic means as defined in claim 14, characterised in that a protruding fold or like gripping point (11) has been formed at the centre of the rip tag (7), at the tip of the sheath (1), the user being able to release the rip tag from the fixing strips (2) by pulling at the fold.

17. A hygienic means as defined in claim 2, characterised in that the end (12) of the rip tag (7) extends over the adhesive surface (3) of the fixing strip (2), forming a gripping point allowing the user to release the rip tag from the fixing strip by pulling at this gripping point.

18. A hygienic means as defined in claim 3, characterised in that the end (12) of the rip tag (7) extends over the adhesive surface (3) of the fixing strip (2), forming a gripping point allowing the user to release the rip tag from the fixing strip by pulling at this gripping point.

19. A hygienic means as defined in claim 12, characterised in that the end (12) of the rip tag (7) extends over the adhesive surface (3) of the fixing strip (2), forming a gripping point allowing the user to release the rip tag from the fixing strip by pulling at this gripping point.
